(19) United States
(12) Patent Application Publication
(10) Pub. No.: US 2005/0228024 A1
(43) Pub. Date: Oct. 13, 2005

(54) Pharmaceutical compositions comprising a benzofuran derivative and their use for the treatment of attention deficit/hyperactivity disorder

(76) Inventor: Peter Haynes Hutson, Saffron Walden (GB)

Correspondence Address: MERCK AND CO., INC, P O BOX 2000, RAHWAY, NJ, 07065-0907, US

(21) Appl. No.: 10/509,604

(22) PCT Filed: Mar. 21, 2003
(86) PCT No.: PCT/GB03/01237

§ 371 Date: Sep. 27, 2004

(30) Foreign Application Priority Data
Mar. 26, 2002 (GB) 0207139.7

(51) Int. Cl. A61K 31/4433

(52) U.S. Cl. 514/337

(57) ABSTRACT

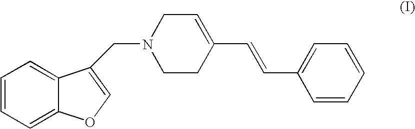

The compound of formula (I): or a pharmaceutically acceptable salt thereof, especially the mesylate salt, is of use in a method for the treatment and/or prevention of attention-deficit/hyperactivity disorder (ADHD).

This invention relates to the use of a particular heteroaromatic compound. More particularly, the invention is concerned with the use of a (1,2,3,6-tetrahydropyridin-1-yl)methyl substituted benzofuran derivative which is a selective antagonist of the dopamine D₄ receptor subtype within the brain and is therefore of benefit in the treatment and/or prevention of attention-deficit/hyperactivity disorder (ADHD).

ADHD is a condition characterised by inattentive, impulsive hyperactive behaviour, and affects some 6% of school age boys in the USA. Although primarily affecting children, in some cases the symptoms persist into adulthood. Several recent studies have implicated the dopamine D₄ receptor in the etiology of ADHD (see, for example, Zhang et al., Neuropsychopharmacology, 2001, 25, 624632, and references therein).

EP-A-1177792 relates to the use of a dopamine D₄ receptor ligand in the treatment or prevention of a novelty-seeking disorder, including attention deficit disorder with hyperactivity disorder. There is in that publication, however, no disclosure nor any suggestion of employing the specific benzofuran derivative of formula I as depicted below, or a pharmaceutically acceptable salt thereof, in the therapy of ADHD.

WO 02/072029, published on 19 Sep. 2002, describes and claims a method of inhibiting motor hyperactivity in a mammal exhibiting the symptoms of ADHD, which comprises administering thereto a compound selected from a list of known dopamine D₄ receptor antagonists. That list does not, however, include the compound of formula I as depicted below or a pharmaceutically acceptable salt thereof.

U.S. Pat. No. 5,665,722 discloses a class of substituted benzofuran derivatives which are selective dopamine D₄ receptor antagonists and which are said to be useful in the treatment of schizophrenia.

According to the present invention, there is provided a method of treating or preventing ADHD comprising administering to a subject in need thereof a therapeutically-effective amount of the compound of formula I: 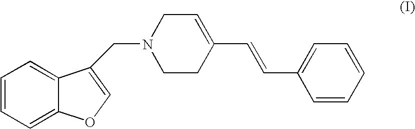
or a pharmaceutically acceptable salt thereof.

Many of the known dopamine D₄ receptor antagonists, e.g. L-745,870 (cf. WO 02/072029), incorporate an indole or aza-indole ring system into their molecular structure, and are accordingly susceptible to being metabolised by a retro-Mannich mechanism, leading to the formation of potentially toxic by-products, e.g. covalent glutathione adducts. The molecular structure of the benzofuran derivative of formula I above, meanwhile, is devoid of an indole or aza-indole ring system; use of this compound in the therapy of ADHD is therefore advantageous, in that there is consequently no possibility of metabolism by a retro-Mannich route.

In one embodiment of the present invention, the subject is a human male. In this embodiment, the subject is typically a human male aged 5-18 years, preferably aged 12-18 years.

The method of treatment according to the invention typically comprises administering to the subject a tablet containing from 1 to 100 mg of the compound of formula I or pharmaceutically acceptable salt thereof once, twice, three times or four times a day. Preferably, the tablet contains from 2 to 50 mg, more preferably from 5 to 25 mg, of the compound of formula I or pharmaceutically acceptable salt thereof, and is administered once or twice a day. In a particular embodiment, a tablet containing 15 mg of the compound of formula I or pharmaceutically acceptable salt thereof is administered once a day.

The method of treatment according to the invention may be used for treatment of ADHD which is of the combined type, or which is of the predominantly inattentive type, or which is of the predominantly hyperactive-impulsive type.

There is further disclosed the use of the compound of formula I or a pharmaceutically acceptable salt thereof for the manufacture of a medicament for treatment or prevention of ADHD.

For use in medicine, the salts of the compound of formula I will be pharmaceutically acceptable salts. Other salts may, however, be useful in the preparation of the compound of use in the invention or of its pharmaceutically acceptable salts. Suitable pharmaceutically acceptable salts of the compound of use in this invention include acid addition salts which may, for example, be formed by mixing a solution of the compound of use in the invention with a solution of a pharmaceutically acceptable acid such as hydrochloric acid, sulphuric acid, fumaric acid, maleic acid, succinic acid, acetic acid, benzoic acid, oxalic acid, citric acid, tartaric acid, methanesulphonic acid, carbonic acid or phosphoric acid. Examples of preferred salts include the methanesulphonate (mesylate) salt.

The medicaments relevant to the invention are typically pharmaceutical compositions comprising the compound of formula I, or pharmaceutically acceptable salt thereof, in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the compositions may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of the compound of formula I, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of use in the present invention. Favoured unit dosage forms contain from 1 to 100 mg, for example 1, 2, 5, 10, 15, 25, 50 or 100 mg, of the active ingredient. The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions relevant to the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of ADHD, a suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day.

In order to alleviate the symptoms of ADHD without causing sedation or extrapyramidal side-effects, the dosage level of the compound of formula I may be selected such that the dose administered is effective in substantially completely blocking the dopamine D₄ receptor subtype in human brain whilst displaying no or negligible dopamine D₂ receptor subtype occupancy. A suitable dosage level in this regard is about 0.001 to 5.0 mg/kg per day, more particularly about 0.005 to 1.0 mg/kg per day, and especially about 0.01 to 0.5 mg/kg per day.

### EXAMPLE 1

Use of the Compound of Formula I for Treatment of ADHD

The mesylate salt of the compound of formula I is prepared as described in Example 1 of GB 2,306,471. Tablets comprising 15 mg of this active ingredient are prepared by conventional means and a single tablet is administered once a day to a subject suffering from, or prone to, ADHD.

1-4. (canceled)

5. A method for the treatment of attention-deficit/hyperactivity disorder comprising administering to a subject in need thereof a therapeutically effective amount of the compound of formula I: 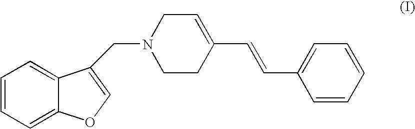
or a pharmaceutically acceptable salt thereof.

6. The method of claim 5 wherein the mesylate salt of the compound of formula I is administered to the subject.

7. The method of claim 5 wherein the subject is a human male.

8. The method of claim 7 wherein the subject is a human male aged 5-18 years.

9. The method of claim 5 wherein the attention-deficit/hyperactivity disorder is of the combined type.

10. The method of claim 5 wherein the attention-deficit/hyperactivity disorder is of the predominantly inattentive type.

11. The method of claim 5 wherein the attention-deficit/hyperactivity disorder is of the predominantly hyperactive-impulse type.

12. A method for the prevention of attention-deficit/hyperactivity disorder comprising administering to a subject in need thereof a therapeutically effective amount of the compound of formula I: 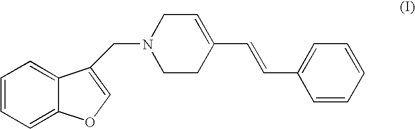
or a pharmaceutically acceptable salt thereof.

13. The method of claim 12 wherein the mesylate salt of the compound of formula I is administered to the subject.

14. The method of claim 12 wherein the subject is a human male.

15. The method of claim 14 wherein the subject is a human male aged 5-18 years.

16. The method of claim 12 wherein the attention-deficit/hyperactivity disorder is of the combined type.

17. The method of claim 12 wherein the attention-deficit/hyperactivity disorder is of the predominantly inattentive type.

18. The method of claim 12 wherein the attention-deficit/hyperactivity disorder is of the predominantly hyperactive-impulse type.
